(19) United States
(12) Patent Application Publication
(10) Pub. No.: US 2004/0181203 A1
(43) Pub. Date: Sep. 16, 2004

(54) Skin treatment method and apparatus for sustained transdermal drug delivery

(76) Inventors: Michel J.N. Cormier, Mountain View, CA (US); Joseph C. Trautman, Sunnyvale, CA (US); Hyunok L. Kim, Chino Hills, CA (US); Ahmad P. Samiee, Sunnyvale, CA (US); Armand P. Neukermans, Portola Valley, CA (US); Bruce P. Edwards, Palo Alto, CA (US); Wai-Loong Lim, Menlo Park, CA (US); Andrew I. Poutiatine, Menlo Park, CA (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/794,637

(22) Filed: Mar. 5, 2004

Related U.S. Application Data
(63) Continuation of application No. 09/733,305, filed on Dec. 8, 2000, now abandoned.
(60) Provisional application No. 60/172,704, filed on Dec. 10, 1999.

(51) Int. Cl. A61M 31/00

(52) U.S. Cl. 604/500; 604/46; 604/289

(57) ABSTRACT

An expandable skin stretching device includes skin engaging opposite ends that contact the skin surface on opposite sides of an area of the skin surface having micropathways therein. When the skin stretching device is expanded the skin between the opposite ends is placed under tension, i.e., stretched. The skin is placed under a tension of about 0.01 to about 10 megapascals, preferably about 0.05 to about 2 megapascals. The skin stretching can be unidirectional or multi-directional. Alternative embodiments of skin stretching devices use suction or normal force to stretch the skin. Stretching the skin helps keep the micropathways open for enhanced agent (e.g., drug) delivery through the micropathways. The stretching also delays micropathway closure.

### TECHNICAL FIELD

The present invention relates to transdermal agent delivery. More particularly, this invention relates to the transdermal delivery of macromolecular agents such as polypeptides, proteins, oligonucleotides and polysaccharides. The present invention relates to devices which have microprotrusions to pierce the outermost layer of a body surface (e.g., the skin) to enhance the transdermal flux of the agents during transdermal delivery.

### BACKGROUND ART

Interest in the percutaneous or transdermal delivery of peptides and proteins to the human body continues to grow with the increasing number of medically useful peptides and proteins becoming available in large quantities and pure form. The transdermal delivery of peptides and proteins still faces significant problems. In many instances, the rate of delivery or flux of polypeptides through the skin is insufficient to produce a desired therapeutic effect due to the low transdermal permeability coefficient of macromolecules and the binding of the polypeptides to the skin. In addition, polypeptides and proteins are easily degraded during and after penetration into the skin, prior to reaching target cells. Likewise, the passive transdermal flux of many low molecular weight compounds is too limited to be therapeutically effective.

One method of increasing the transdermal delivery of agents relies on the application of an electric current across the body surface referred to as “electrotransport.” “Electrotransport” refers generally to the passage of a beneficial agent, e.g., a drug or drug precursor, through a body surface, such as skin, mucous membranes, nails, and the like. The transport of the agent is induced or enhanced by the application of an electrical potential, which results in the application of electric current, which delivers or enhances delivery of the agent. The electrotransport of agents through a body surface may be attained in various manners. One widely used electrotransport process, iontophoresis involves the electrically induced transport of charged ions. Electroosmosis, another type of electrotransport process, involves the movement of a solvent with the agent through a membrane under the influence of an electric field. Electroporation, still another type of electrotransport, involves the passage of an agent through pores formed by applying a high voltage electrical pulse to a membrane. In many instances, more than one of these processes may be occurring simultaneously to a different extent. Accordingly, the term “electrotransport” is given herein its broadest possible interpretation, to include the electrically induced or enhanced transport of at least one charged or uncharged agent, or mixtures thereof, regardless of the specific mechanism or mechanisms by which the agent is actually being transported. Electrotransport delivery generally increases agent delivery and reduces polypeptide degradation during transdermal delivery.

Another method of increasing the agent flux involves pre-treating the skin with, or co-delivering with the beneficial agent, a skin permeation enhancer. A permeation enhancer substance, when applied to a body surface through which the agent is delivered, enhances its flux therethrough such as by reducing the electrical resistance of the body surface to the passage of the agent (i.e., during transdermal electrotransport delivery), increasing the permselectivity and/or permeability of the body surface, creating hydrophilic pathways through the body surface, and/or reducing the degradation of the agent.

There also have been many attempts to mechanically penetrate or disrupt the skin in order to enhance the transdermal flux, such as, U.S. Pat. No. 3,814,097 issued to Ganderton, et al., U.S. Pat. No. 5,279,544 issued to Gross, et al., U.S. Pat. No. 5,250,023 issued to Lee, et al., U.S. Pat. No. 3,964,482 issued to Gerstel, et al., Reissue 25,637 issued to Kravitz, et al., and PCT Publication Nos. WO 96/37155, WO 96/37256, WO 96/17648, WO 97/03718, WO 98/11937, WO 98/00193, WO 97/48440, WO 97/48441, and WO 97/48442. These devices use piercing elements of various shapes and sizes to pierce the outermost layer (i.e., the stratum corneum) of the skin. The piercing elements disclosed in these references generally extend perpendicularly from a thin, flat member, such as a pad or sheet. The piercing elements in some of these devices are extemely small, some having dimensions (i.e., a microblade length and width) of only about 25-400 μm and a microblade thickness of only about 5-100 μm. These tiny stratum corneum piercing/cutting elements are meant to make correspondingly small microslits/microcuts in the stratum corneum for enhanced transdermal agent delivery therethrough. In many instances, the microslits/microcuts in the stratum corneum have a length of less than 150 μm and a width which is substantially smaller than their length.

### DESCRIPTION OF THE INVENTION

It has now been discovered that in the case of human skin, the small microslits/microcuts are quickly closed and sealed by the skin's natural healing processes. In some instances, the enhancement in transdermal agent flux provided by the microslits is completely eliminated within several hours of making the microslits. Thus, there is a need for a method and apparatus which can prevent, or at least delay the skin's natural healing processes in order to allow enhanced transdermal delivery of agents over longer periods of time (e.g., longer than about 1 hour).

The present invention provides enhanced and sustainable agent flux by applying tension to skin at the application site to maintain and/or enlarge pathways created by tiny skin penetrating elements. This enhanced agent flux is provided by applying a tension in the range of about 0.01 M Pa to about 10 M Pa, preferably about 0.05 M Pa to about 2 M Pa, to the skin after penetration of the body surface with the skin penetrating elements and holding the stretched condition during delivery of the agent. Applying tension to the body surface with the device of the present invention maintains and/or enlarges the pathways made in the outermost layer of the body surface. The tension also delays the closing and sealing of the pathways caused by the skin's natural healing process. The terms “applying tension” and “stretching” are used synonymously herein when referring to the pierced body surface.

In one aspect of the present invention, a skin perforating device penetrates the outermost layer of the skin, i.e., stratum corneum, with a plurality of microprotrusions to form pathways in the form of microslits or microcuts through which an agent such as a drug can be introduced, i.e., delivered. The term “microprotrusions” as used herein refers to very tiny skin piercing elements, typically having a length of less than 500 μm, a width of less than 400 μm and a thickness of 5 to 100 μm, which make a correspondingly sized microcut or microslit in the skin. The device then uses stretching elements which engage the surface of the skin, such as with adhesive, and create opposing forces across the surface of the skin so as to create tension at the skin surface between the skin stretching elements. With the skin held in tension, the pathways through the skin are held open during treatment to facilitate sustainable and increased flux. The stretched skin provides improved flux particularly if electrotransport is used for sustained agent delivery through the skin.

In another aspect of the invention, the apparatus comprises an expandable device with skin engaging portions which in use stretches the patient's skin and a relatively thin, flexible sheet, which in use is adapted to be placed in substantially parallel relation with the body surface to be pierced. The sheet has a plurality of microprotrusions extending from a body proximal side of the sheet for forming the pathways, i.e., microcuts/microslits in the outermost layer of the body surface. Preferably, the microprotrusions have a blade-like configuration and the expandable device is oriented to stretch the skin in a direction that is transverse to the plane of the microprotrusions.

The device of the present invention can be used in connection with agent delivery, and in particular, transdermal drug delivery. Delivery devices for use with the present invention include, but are not limited to, electrotransport devices, passive devices, osmotic devices, and pressure-driven devices.

The present invention also includes a method of maintaining pathways in an outermost layer of body tissue (e.g., the stratum corneum layer of the skin), open during transdermal agent delivery. The method includes the steps of penetrating a body surface with a plurality of microprotrusions to create pathways in the body surface, contacting the body surface with a stretching device, and actuating the stretching device to stretch the body surface and expand the cross sectional area of the pathways.

### BRIEF DESCRIPTION OF THE DRAWINGS

Many objects and advantages of the present invention will be apparent to those skilled in the art when this specification is read in conjunction with the attached drawings, wherein like reference numerals are applied to like elements, and wherein:

FIG. 1 is a top plan view of a skin stretching device of the present invention applied to skin having a plurality of microslits therein;

FIG. 2 is a cross-sectional view of the device shown in FIG. 1 taken along line 2-2 of FIG. 1;

FIG. 3 is a diagrammatic top view of the pathways made in a body surface while being stretched in accordance with the present invention;

FIG. 4 is a perspective view of a first embodiment of a body surface stretching device in accordance with the present invention;

FIG. 5 is a cross-sectional view of the device shown in FIG. 4, taken along line 5-5 of FIG. 4;

FIG. 6 is an enlarged perspective view of a portion of the device shown in FIGS. 4 and 5;

FIGS. 7-9 illustrate the operation of a second embodiment of a body surface stretching device in accordance with the present invention;

FIGS. 10 and 11 illustrate operation of a third embodiment of a body surface stretching device in accordance with the present invention;

FIG. 12 is a side cross-sectional view of a fourth embodiment of a body surface stretching device in accordance with the present invention;

FIG. 13 is a side cross-sectional view of a fifth embodiment of a body surface stretching device in accordance with the present invention;

FIG. 14 is an exploded perspective view of an alternate embodiment of a microprotrusion array which can be used with the present invention; and

FIG. 15 is a graph of tension or force applied versus skin strain.

### MODES FOR CARRYING OUT THE INVENTION

Each of the embodiments illustrated in the Figures for the present invention stretch the body surface or skin 30 after pathways 31 have been formed by piercing the body surface with a plurality of microprotrusions 34. The stretching or tensioning of the skin maintains the pathways open and preferably enlarges the cross sectional area of the pathways 31 in the skin. Thus, the present invention involves making small cuts, slits, or holes called pathways in the outermost layer of the body surface to a limited depth by treating the body surface with a device having microprotrusions. Following microprotrusion treatment, the treated body surface is subjected to a predetermined amount of tension (i.e., stress). The skin is stretched in one or more directions by a skin stretching device. The skin stretching/tensioning devices according to the present invention apply a predetermined amount of tension (i.e., stress) in the range of from about 0.01 M Pa to about 10 M Pa (M Pa=megapascal=1×10⁶ pascals). The preferred range of tension/stress is about 0.05 M Pa to about 2 M Pa. The amount of skin strain resulting from a given tension varies between individuals depending upon skin characteristics, such as the age of the patient, the location of the application on the patient's body, and the tensioning direction. Therefore, in order to adapt to individual characteristics, the skin tensioning devices according to the present invention preferably are designed to provide a given tension (stress) rather than a given strain. In general, for these stress or tension ranges, the applied skin strain is within about 5 to 60% and most preferably within about 10 to 50%. Strain is the amount of skin stretch per unit length of skin and is defined as the change in length of skin caused by stretching divided by the length of the skin in a non-stretched state. The strain of skin can be expressed mathematically by the following equation:

Strain=(Iₑₓₜ−Iₙₒₙ₋ₑₓₜ)+Iₙₒₙ₋ₑₓₜ

wherein:

Iₑₓₜ is the length of a sample of skin in a stretched state; and

Iₙₒₙ₋ₑₓₜ is the length of the skin sample in a non-stretched state.

Stretching the skin keeps the pathways open for longer periods, i.e., pathway closure is postponed. An agent delivery device is placed in contact with the treated and stretched region of the body surface to deliver an agent therethrough.

The present invention improves the agent flux by stretching the outermost layer(s) of the body surface after the pathways have been made. For elongated pathways in the shape of slits, the direction of stretching is transverse to the length of the slits so as to enlarge the cross-sectional area of the slits in the body surface. For circular pathways, the direction of stretching is preferably multi-directional to enlarge the cross-sectional area of the circular holes. For other shaped pathways the direction of stretching may be bidirectional or multidirectional as necessary in order to enlarge the cross-sectional area of the pathways. With each of the embodiments of the present invention, the skin is stretched either after puncturing or during and after puncturing. After penetration by the plurality of microprotrusions 34, the skin is maintained under tension created by stretching the skin to maintain the pathways open through an administration period.

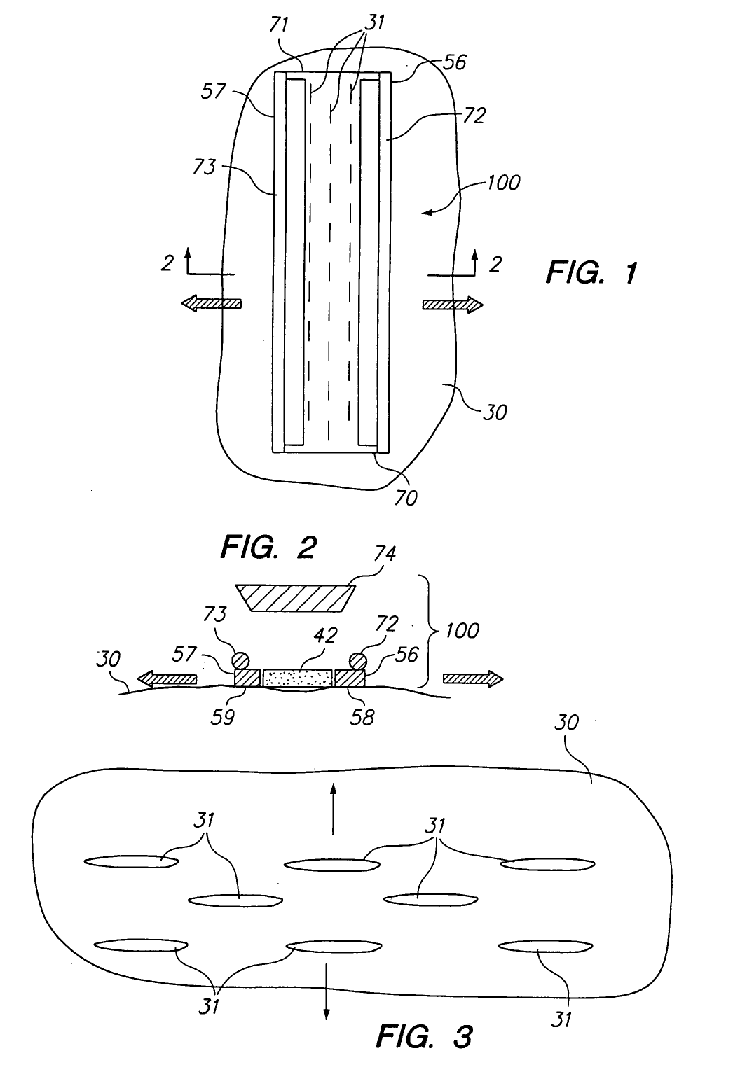

The devices of the present invention, shown in FIGS. 1, 2, and 4-13, are designed to provide a tension (i.e., stress) of about 0.01 to about 10 M Pa, and preferably from about 0.05 to about 2 M Pa, to the skin.

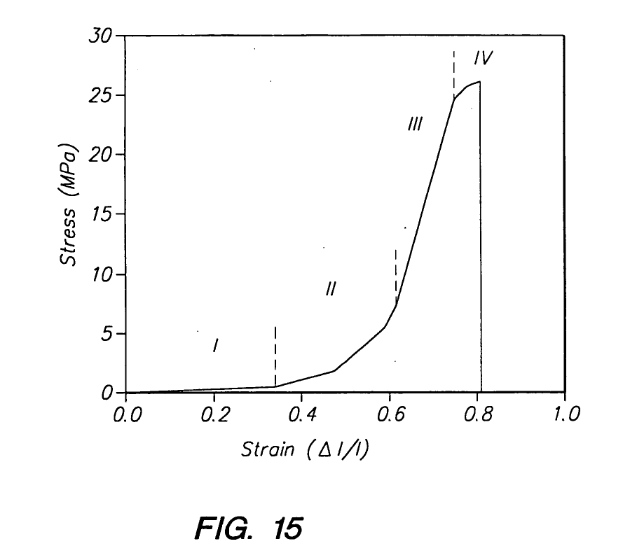

FIG. 15 illustrates the typical stress-strain curve for an in vitro tensile test on excised mammalian skin. In phase I there is rapid extension of skin under low load. In phase II there is rapid stiffening of skin followed by phase III in which the skin has stiff behavior. If the skin is tensioned to a degree that reaches phase IV, skin tearing and rupture occurs. For effective skin stretching according to the present invention, it is desirable to tension to a degree that results in phase II or III strain but not phase IV strain. FIG. 15 illustrates the typical stress-strain curve for unpierced skin, however, the curve may vary somewhat for skin which has been punctured by an array of microprotrusions.

The device of the present invention is for use in the percutaneous administration of an agent. The terms “substance”, “agent”, and “drug” can be used interchangeably and broadly include physiologically or pharmacologically active substances for producing a localized or systemic effect or effects in mammals, including humans and primates, avians, valuable domestic household, sport or farm animals, or for administering to laboratory animals such as mice, rats, guinea pigs, and the like.

The major barrier properties of the skin, such as resistance to agent permeation, reside with the outermost layer of the skin, i.e., stratum corneum. The inner division, i.e., the backing layers, of the epidermis generally comprises three layers commonly identified as stratum granulosum, stratum malpighii, and stratum germinativum. There is essentially little or no resistance to transport or to absorption of an agent through these layers. Therefore, for enhancing transdermal flux the microprotrusions used to create pathways in the body surface in accordance with the present invention need only penetrate through the stratum corneum in order for the agent to be transdermally delivered with little or no resistance through the skin.

The embodiments of the invention utilize a plurality of microprotrusions to pierce the body surface. Preferably, the microprotrusions are in the form of a microprotrusion array as shown in FIG. 6 with a plurality of blade type microprotrusions 34 extending outwardly from one surface of a thin, compliant member or sheet 36. The sheet 36 of the FIG. 6 embodiment is oriented during use with a main surface parallel to the patient's body surface 30. The microprotrusions 34 extend from a skin proximal edge of the sheet 36 and are oriented with the planar surfaces of the microprotrusing blades perpendicular to the patient's body surface.

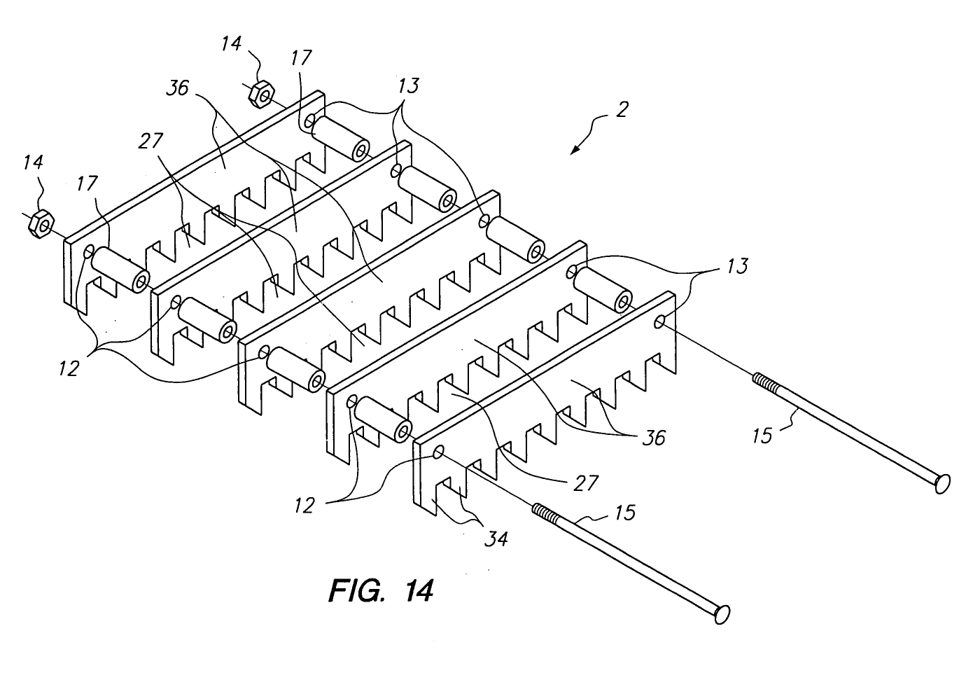

A particularly preferred configuration for the device is illustrated in FIG. 14 and comprises a plurality of individual sheet members 36 stacked together to form a microprotrusion device 2. Each thin sheet 36 in use is oriented perpendicular to the patient's body surface 30. The sheets 36 each have a plurality of microprotrusions 34 in the same plane as the sheet 36 which extend outward from a body proximal edge 38 of the sheet 36 for penetrating the body surface 30. Each of the sheet members 36 has a pair of holes 12, 13 through which bolts 15 are inserted. Spacers (e.g., tubes) 17 are positioned between each adjacent pair of sheet members 36 to form voids 27 therebetween. The spaced sheet members 36 are held together as a unit by passing the bolts 15 through the sheet members and spacers 17 and securing nuts 14 on the ends of the bolts, or using other known fasteners. The voids 27 can be filled with a reservoir matrix material (e.g., a gel) adapted to contain the beneficial agent to be delivered. Those skilled in the art will appreciate that spacers 17 having other than tube-like configurations (e.g., square or rectangular blocks) can also be used to provide voids 27 between the agent reservoir 42 (i.e., the agent reservoir contained in the voids 27 ) and the skin. Furthermore, more than two sets of bolts 15, or other fastening pins, may be used to secure the sheet members 36 and spacers 17 together.

Although either of these two embodiments are preferred because of the many advantages they afford over the prior art, the present invention can be used with other known microprotrusion arrays, for example, those described in U.S. Pat. No. 5,279,544 issued to Gross et al.; U.S. Pat. No. 3,964,482 issued to Gerstel et al.; U.S. Pat. No. 5,250,023 issued to Lee et al.; Reissue 25,637; U.S. Pat. No. 5,312,456 issued to Reed et al.; and disclosed in PCT Publication Nos. WO97/48440; WO96/37256; WO97/03718; WO98/11937; and WO98/00193, which are incorporated herein by reference in their entirety. The sheet member shown in FIG. 14 may alternatively be coiled in a loose spiral or folded in a serpentine configuration. Alternatively, a plurality of cylindrical sheet members may be placed in concentric circles.

Either of the embodiments of microprotrusion arrays shown in FIGS. 6 and 14 can be embodied in separate devices that are used to pretreat the patient's body surface and then removed so that the skin stretching and drug reservoir device of the present invention can be applied to the pretreated area. Other pretreatment skin perforating devices can be used in conjunction with the present invention. For example, the device described in U.S. Pat. No. 5,611,806 issued to Jang, which is incorporated herein by reference in its entirety, comprises a plurality of alternately disposed needle disks and spacers which are combined together for rotational movement as a unit. The device is rolled over the skin to form a plurality of fissures.

FIGS. 1 and 2 illustrate a device 100 for stretching a body surface 30 having a plurality of preformed pathways 31 in the body surface. The pathways 31 may be formed with the microprotrusions 34 as shown in FIGS. 6 or 14 or with a rolling tool, for example as described in U.S. Pat. No. 5,611,806 issued to Jang. The device 100 has an agent delivery reservoir 42 located between a pair of elongated opposing skin stretching members 56 and 57 held together by expandable elements 70 and 71 (see FIG. 1 in which reservoir 42 is removed to show the fissures 31). The elongated members 56 and 57, as clearly shown in FIG. 1, are arranged so that the axes of members 56 and 57 are roughly parallel with the lengths (i.e., the longer dimension) of the slits 31. The elongated opposing skin stretching members 56 and 57 have skin engaging surfaces 58 and 59, e.g., adhesive-coated surfaces 58 and 59, which engage and adhere to the body surface 30. A pair of cylindrical elements 72 and 73 are attached to the skin distal sides of the members 56 and 57, respectively. A wedge 74 is pressed vertically, i.e., toward the skin, between the cylindrical elements 72 and 73 which displaces the members 56 and 57 in the direction of the arrows. This displacement of members 56 and 57 creates transverse tension in the body surface 30 between the members 56 and 57 and enlarges the cross-sectional area of the pathways 31 as illustrated in FIG. 3 to provide increased open area for agent transport. A locking mechanism (not shown in the Figures) can be provided within device 100 to lock the wedge into place between cylindrical elements 72 and 73 in their displaced (i.e., skin stretched) position. The agent is transported from the reservoir 42 through the open pathways 31. The tension can be relieved and reapplied several times to reopen the pathways 31, if necessary, during transdermal agent delivery. Likewise, the wedge 74 can be inserted farther at a subsequent time to increase the skin tension progressively over time.

As best illustrated in FIG. 1, for a given area of skin 30 to be stretched, the expandable skin stretching members 56 and 57 are preferably in an elongated configuration, i.e., they preferably have a length much greater than the distance separating members 56 and 57. In other words, the distance separating the skin stretching members 56 and 57 is small compared to the length of the members 56 and 57. In this manner, a relatively large area of skin 30 can be stretched by relatively small lateral movement of the members 56 and 57 in the direction of the arrows shown in FIGS. 1 and 2. This configuration is likewise preferred from the standpoint of adhering members 56 and 57 to the surface of skin 30 since the adhesive forces are highest per unit area in this configuration.

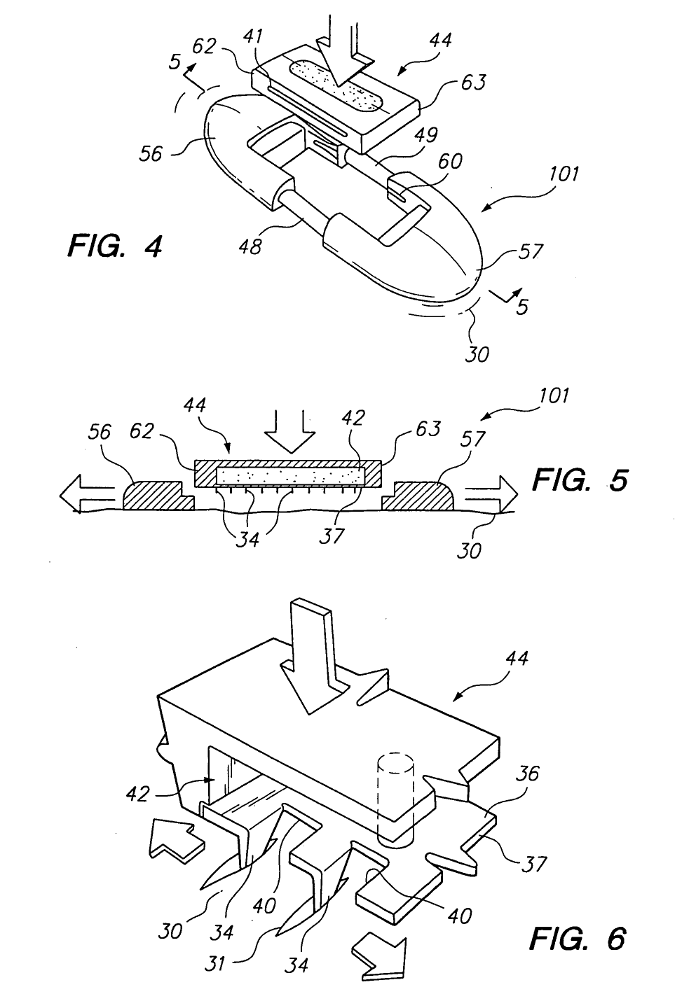

In the embodiment shown in FIGS. 4 and 5, a stretching device 101 uses biasing members such as springs (not shown in FIGS. 4 and 5 ) housed in cylindrical members 48 and 49 to stretch the body surface 30 after pathways have been created with the microprotrusions 34 on the cartridge 44. The expandable device 101 has an adhesive coating on the skin contacting surfaces of opposing skin stretching members 56 and 57. The adhesive coating is initially protected by a release liner (not shown) which is removed just prior to placing the device 101 on the skin. The opposite ends 56 and 57 of the device 101 are initially held together manually or mechanically with a retainer such that the biasing members located within the cylindrical members 48 and 49 are in compression. The patient removes the release liner and applies the device 101 to a portion of the skin surface 30 such that the adhesive on the underside holds the device 101 to the patient's skin surface 30. A snap-in cartridge 44 having an array of microprotrusions 34 on a skin contacting surface and a reservoir 42 therein is then snapped into the opening between the two opposing ends 56 and 57 such that protrusions 41 on each side (only one shown in FIG. 4) of the housing 44 lock into the indentations 60 (only one shown) in the device 101 as the microprotrusions 34 pierce the outermost layer of the skin surface 30. Then the patient removes a disposable retainer (not shown) from the device 101 to release the biasing members inside the cylindrical members 48 and 49 which are in compression so that the device 101 expands to stretch the skin.

In an optional embodiment not illustrated, the ends 62 and 63 of the cartridge 44 form a wedge to match inclined surfaces in the device 101 so that when the cartridge 44 is snapped into the expandable device 101, the ends 62 and 63 maintain the skin in a stretched condition after penetration of the body surface by the microprotrusions 34. As seen in FIG. 6, the stretching of the body surface 30 in a direction transverse to the length (i.e., the longer dimension) of the slits 31 (and also transverse to the plane of the microprotrusions 34 ) holds the slits 31 open during agent delivery.

FIGS. 5 and 6 have been illustrated with a snap in cartridge 44 containing both the microprotrusions 34 and the reservoir 42. When the cartridge 44 having the microprotrusions 34 and reservoir 42 is used, the skin is preferably stretched prior to application of the microprotrusions. Alternatively, the skin may be pretreated to form pathways 31 prior to stretching and application of an agent delivery cartridge. The stretching of the skin 30 is maintained through an agent (e.g., drug) administration period.

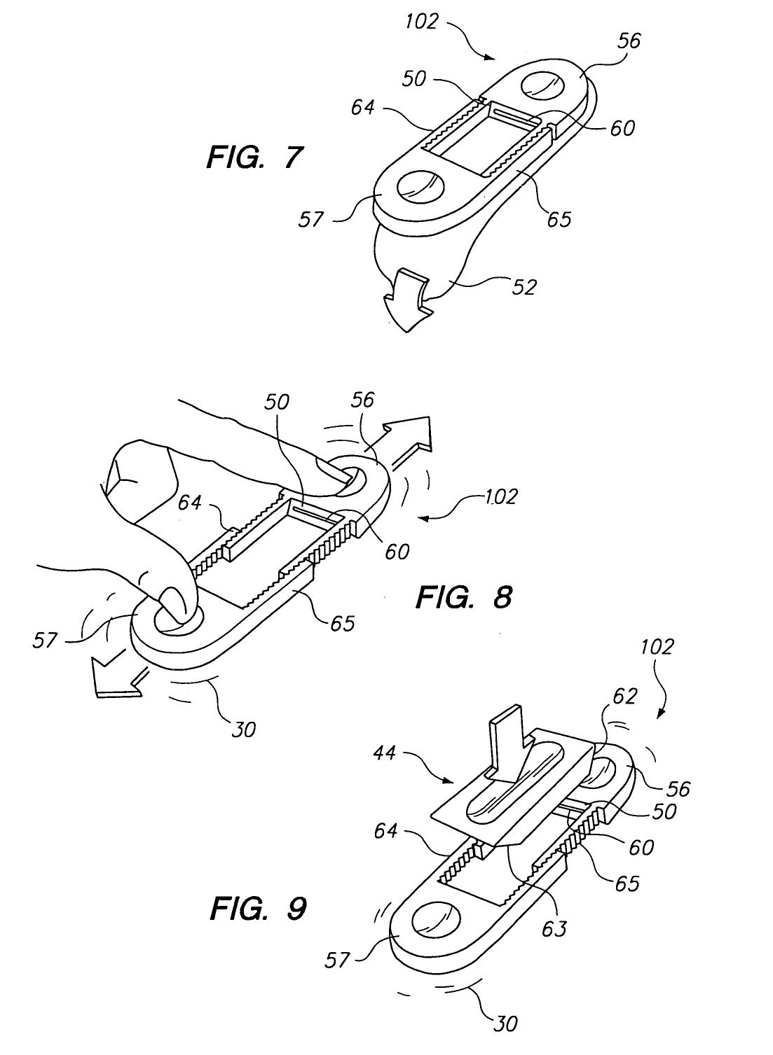

In the embodiment illustrated in FIGS. 7 through 9, an expandable device 102 is manually actuated by the patient, but essentially retains the same characteristics of operation as described with respect to the earlier embodiments, in that the skin is stretched after puncturing. With respect to this embodiment, pathways are made in the body surface using one of the pretreatment devices described previously. Then the release liner 52 is removed to expose the adhesive on the underside of each of the ends 56 and 57 of the device 102, and the device is placed on the patient's pretreated body surface. The patient or another person then stretches the body surface 30 by spreading apart the ends 56 and 57 of the device 102 (as shown in FIG. 8), a predetermined amount which achieves a skin tension in the range of about 0.01 to about 10 M Pa, and preferably about 0.05 to about 2 M Pa. The ratcheted sides 64 and 65 on the expandable device 102 allow the device to maintain its expanded position after the patient or another medical technician has removed their hand from the device. The snap-in cartridge 44 having no microprotrusions is then pressed down with a load applied normal to the body surface as described with respect to the earlier embodiment. If the snap-in cartridge housing 44 has the optional wedge shape, then the insertion of the housing 44 into device 102 can be used to achieve the predetermined level of skin stretching. The snap in cartridge housing 44 has a drug-containing reservoir which provides transdermal drug delivery. It is within the scope of the present invention for the cartridge 44 to have microprotrusions 34 on the body surface thereof to create additional pathways.

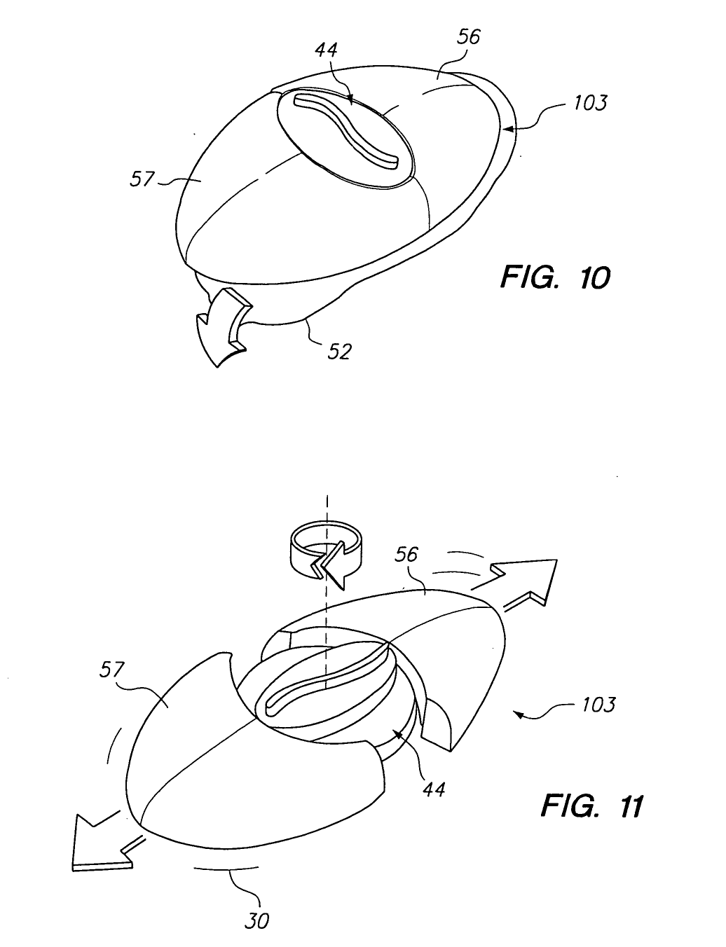

According to a further alternative embodiment a stretching device 103 illustrated in FIGS. 10 and 11 is operated with a rotational motion, rather than a translational motion, in order to move the opposing ends 56 and 57 a predetermined distance apart which achieves a skin tension in the range of about 0.01 to about 10 M Pa, and preferably about 0.05 to about 2 M Pa. The device 103 is an integral unit, rather than having a separate cartridge 44 with the agent reservoir therein, as in devices 100-102. The patient or another person pretreats the body surface 30 to create the pathways therein then removes the release liner 52 from the underneath side of the device 103 to expose the adhesive on the skin proximal surface of each end 56 and 57, and places the device 103 on the patient's body surface 30. The reservoir housing 44 is then rotated to bring the agent reservoir (not shown) into contact with the body surface 30, and due to its elliptical shape, forces the ends 56 and 57 of the expandable device 103 apart to stretch the body surface 30 transverse to the length of the pathways created by the pretreatment.

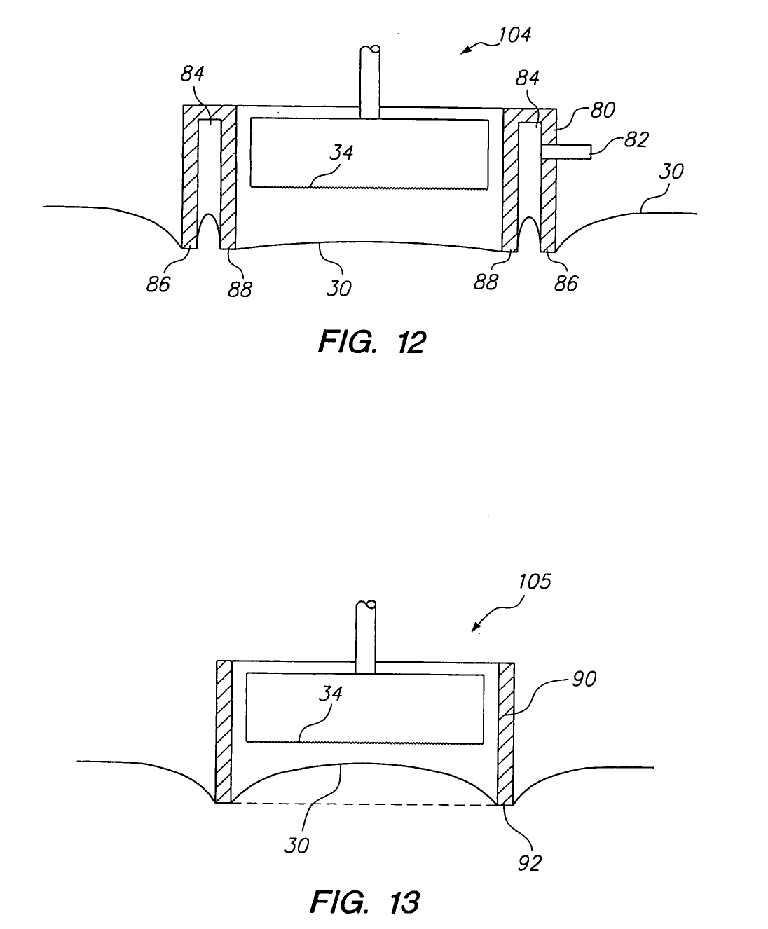

A fifth embodiment of the invention is illustrated in FIG. 12. In this embodiment, the device 104 includes a suction member 80 of a rectangular, square, circular, or other shape in plan view connected to a tube 82 for drawing a suction. The suction member 80 has a suction channel 84. For multi-directional stretching, the suction channel 84 may be a continuous or substantially continuous channel having a rectangular, circular, oval, or other shape in plan view. For uni-directional stretching, two opposed suction channels 84 may be provided. An outer edge of the suction member 80 includes a lower surface 86 which grips the skin by having a high friction coefficient with respect to the skin. A lower surface 88 of an inner edge of the channel 84 is preferably provided with a low friction surface which allows the skin to slide over the inner surface. The non-slip surface 86 may be an adhesive, while the slip surface 88 may be coated with a lubricant. When a suction is applied to the suction tube 82 a low pressure area is provided within the channel 84 which draws the skin 30 into the channel as shown in FIG. 12, stretching the skin within the opposing surfaces 88. The amount of suction applied within channel 84 will vary depending on the size of device 104. Those persons of ordinary skill in the art can determine the level of suction needed to achieve a skin tension in the range of about 0.01 to about 10 M Pa, and preferably about 0.05 to about 2 M Pa. The skin is treated with an array of microprotrusions 34 either before or after stretching. A cartridge (not shown) is inserted into an interior of the device 104 and placed against the stretched skin for agent delivery.

A sixth embodiment of the invention is illustrated in FIG. 13. The device 105 includes a tubular member 90 having a lower edge 92 which is pressed into the skin surface 30 causing skin in a center of the tubular member to form a dome shape and become tensioned. The amount of skin stretching or tensioning may be controlled by the amount of pressure applied to the tubular member 90. Manually applied pressure is not recommended in this embodiment since the amount of skin tension achieved through manually applied downward pressure applied to member 90 will be variable and difficult to ensure a skin tension in the range of about 0.01 to about 10 M Pa. Thus with this embodiment, a device (not shown) for applying a predetermined downward (i.e., toward the skin) force to the tubular member 90 is recommended. To prevent slipage between the skin and the lower edge 92 of the tubular member 90, a non-slip surface, such as an adhesive may be employed. The array of microprotrusions 34 are then applied before or after tensioning of the skin. A cartridge (not shown) is inserted in a center of the tubular member 90 and placed against the treated skin with enlarged pathways to deliver an agent. The device 105 as shown in FIG. 13 may be cylindrical, square, rectangular, or any other shape in plan view.

A seventh embodiment of the invention which is not illustrated in the figures comprises an elastic strap which is adapted to extend partially, but not completely, around a limb (i.e., an arm, leg or finger) of a patient. The elastic strap has adhesive coated at the ends thereof which adhesive is adapted to be placed in contact with the skin on the limb. A skin site on the limb to be stretched is first identified. The strap is applied by first removing an adhesive liner at one end of the strap and adhering that end of the strap adjacent to the skin site to be stretched. The elastic strap is then extended around the limb, in a condition having a predetermined amount of stretch (i.e., sufficient stretch to achieve a skin tension in the range of about 0.01 to about 10 M Pa, and preferably about 0.05 to about 2 M Pa) and the other end of the strap is adhered adjacent the opposite side of skin site to be stretched. The stretching of the elastic strap stretches the skin site positioned between the two adhered end portions of the strap.

The expandable devices 100 through 105 of any of the embodiments can be held in contact with the body surface 30 in any of a variety of ways, such as but not limited to adhesive, tape, a strap, or an elastic bandage.

In the preferred embodiments, the microprotrusions 34 are microblades as shown in FIGS. 6 and 14. In the embodiment shown in FIG. 6, the sheet member 36 is formed with a plurality of openings 40 adjacent the microprotrusions 34 to permit the transport of agent from an agent reservoir 42 located on the skin distal side of sheet 36 within cartridge 44. In this embodiment, the openings 40 correspond to the portion of the sheet member 36 occupied by each of the microprotrusions 34 prior to the microprotrusions being bent into a position which is substantially perpendicular to the plane of the sheet member 36 as shown.

The preferred configurations for the array of microprotrusions and a connecting medium for delivering agents from the reservoir 42 to the body surface are described in detail in WO 97/48440; WO 97/48441; WO 97/48442; and WO 98/28037 which are incorporated herein by reference in their entirety.

The array of microprotrusions 34 in the various embodiments of the present invention may take on different shapes. The present invention can be used with any known delivery device and is not limited to any particular device. It will be appreciated by those working in the field that the present invention can be used in conjunction with a wide variety of electrotransport systems, as the invention is not limited in any way in this regard, see for example electrotransport systems disclosed in U.S. Pat. No. 5,147,296 issued to Theeuwes et al., U.S. Pat. No. 5,080,646 issued to Theeuwes et al., U.S. Pat. No. 5,169,382 issued to Theeuwes et al., U.S. Pat. No. 5,423,739 issued to Phipps et al., U.S. Pat. No. 5,385,543 issued to Haak et al., U.S. Pat. No. 5,310,404 issued to Gyory et al., and U.S. Pat. No. 5,169,383 issued to Gyory et al., and PCT Publication No. WO07/48440, which are incorporated herein by reference in their entirety. Similarly, any known passive transdermal delivery device can be used with the present invention, as the invention is not limited in this regard, see for example passive systems disclosed in U.S. Pat. No. 4,379,454 issued to Campbell et al., U.S. Pat. No. 4,588,580 issued to Gale et al., U.S. Pat. No. 4,832,953 issued to Campbell et al., U.S. Pat. No. 4,698,062 issued to Gale et al., U.S. Pat. No. 4,867,982 issued to Campbell et al., and U.S. Pat. No. 5,268,209 issued to Hunt et al., and PCT Publication No. WO97/48440, which are incorporated herein by reference in their entirety. It will be appreciated by those working in the field that the present invention can also be used in conjunction with a wide variety of osmotic and pressure driven systems, as the invention is not limited to a particular device in this regard, for example see U.S. Pat. No. 4,340,480 issued to Pall et al., U.S. Pat. No. 4,655,766 issued to Theeuwes et al., U.S. Pat. No. 4,753,651 issued to Eckenhoff et al., U.S. Pat. No. 5,279,544 issued to Gross et al., and U.S. Pat. No. 5,242,406 issued to Gross et al., which are incorporated herein by reference in their entirety.

The following example illustrates the utility of the present invention in enhancing transdermal polypeptide flux.

### EXAMPLE

In this experiment, a radio-labelled synthetic model decapeptide was delivered by passive diffusion through the skin of live hairless guinea pigs at skin sites that were pretreated by piercing with an array of microprojections. The purpose of the experiment was to determine whether stretching the skin following the microprojection pretreatment improved flux of the drug through the skin in vivo. The decapeptide had a molecular weight of 1141 daltons and a water solubility of greater than 50 mM over a wide pH range. The decapeptide had been previously found to exhibit insignificant (i.e., less than 0.1 μg/cm² hr) passive diffusional flux through untreated skin.

Twelve hairless guinea pigs were divided into two groups of six. The first group (test group) had skin under tension during transdermal decapeptide delivery. The second group (control group) had skin under normal, relaxed (i.e., no tension) conditions during transdermal decapeptide delivery. Both groups had skin sites under tension when the microprotrusion arrays were applied since previous studies have shown that stretching the skin at the time of microprotrusion application helps the microprotrusions to more consistently pierce the skin. The microprotrusion arrays used in the study had the configuration of a metal sheet 36, with a multiplicity of openings 40 and microprotrusions 34 as shown in FIG. 6. Each array was a stainless steel sheet having a thickness of 0.025 mm (1 mil) having a multiplicity of photoetched and punched microprotrusions extending roughly perpendicular to the plane of the sheet. The sheet had an area of 2 cm², 72 openings and 144 microprojections. The microprojections had a triangular shape with a length 545 μm. In both groups (test and control), the skin of one flank was stretched manually in two directions (⇄ and [symbol] ) when the microprojection arrays were applied. The bi-directional stretching was estimated to achieve a skin tension of between 0.1 and 1 M Pa. The microprotrusion arrays were positioned on the skin by first applying a foam double-sided adhesive ring (diameter 3.8 cm, thickness 0.16 cm with a 2 cm² central opening) on the skin site. Next, the microprotrusion array was layed in the opening, microprotrusion side down. Next, the skin distal side of the microprotrusion array was struck using a spring-loaded applicator. Following microprotrusion piercing, the manually applied stretching tension was released.

In the test group, the adhesive ring was left in place and served to keep the skin under the drug compartment (i.e., the drug formulation-filled opening in the foam ring) in the stretched configuration. After the microprojection array was removed, a hydrogel containing an aqueous solution (pH 6) of the tritiated decapeptide in an hydroxy ethyl cellulose gel 10 mM, 2% HEC) was dispensed into the opening in the foam ring and a plastic cover was applied to the adhesive outer surface of the ring to seal the drug formulation.

In the control group, the microprojection array and the double-sided adhesive foam ring were removed following microprotrusion piercing causing the stretching tension to be released and the skin to return to its normal relaxed (i.e., unstretched) state. An adhesive ring was placed over the skin site in a relaxed state and the same formulation was dispensed into the opening in the foam ring, which was subsequently sealed with a plastic cover.

At 1 and 24 h after applying the decapeptide formulation to the animals' skin, three animals from each group were removed and residual drug washed from the skin. The amount of drug penetrated during these time intervals was determined by measuring urinary excretion of tritium (previous studies had shown that in hairless guinea pigs, 65% of the tritium derived from tritiated decapeptide injected intravenously is excreted in urine). The results (Table 1) show that keeping the microslit skin in a stretched state during passive transdermal decapeptide delivery increases the amount of decapeptide delivered by several fold for up to 24 h.

TABLE 1

| | Mean Decapeptide | | Mean Decapeptide Flux | |
|---|---|---|---|---|
| | Delivered (μg) | | Per Unit Area (μg/cm²h) | |
| Animal | Application Time (h) | | Application Time (h) | |
| Group | 1 h | 24 h | 1 h | 24 h |
| Test | 27.9 ± 3.3 | 94.7 ± 23.7 | 13.9 ± 1.6 | 1.97 ± 0.49 |
| (stretching) | | | | |
| Control | 9.8 ± 0.8 | 23.5 ± 5.9 | 4.9 ± 0.4 | 0.49 ± 0.12 |
| (no stretching) | | | | |

The mean decapeptide flux per unit area was also calculated at the mid-time points of the 1 and 24 hour periods (i.e., at 0.5 hours and 11.5 hours). The results (Table 2) show that the decrease in flux per unit area is less with stretching of the skin compared to no stretching of the skin. These results suggest that stretching of the skin following microprojection piercing and during transdermal drug delivery retards the closure of the microslits in the skin.

TABLE 2

Mean Decapeptide Flux
per Unit Area (μg/cm² h)
Mid-time (h)

| Animal Group | 0.5 h | 12.5 h |
|---|---|---|
| Test (stretching) | 13.9 ± 1.6 | 1.45 ± 0.49 |
| Control (no stretching) | 4.9 ± 0.4 | 0.30 ± 0.12 |

It will be appreciated by those of ordinary skill in the art that the invention can be embodied in other specific forms without departing from the spirit or essential character thereof. The presently disclosed embodiments are therefore considered in all respects to be illustrative and not restrictive. The scope of the invention as indicated by the appended claims rather than the foregoing description, and all changes which come within the meaning and range of equivalence thereof are intended to be embraced therein.

1. A method for delivering an agent through a body surface, comprising:
penetrating the body surface with a plurality of microprotrusions to create micropathways in the body surface;
stretching the penetrated body surface by applying a tension of about 0.01 to about 10 M Pa to the penetrated body surface during flux of the agent therethrough; and
delivering an agent from a reservoir positioned in agent transmitting relation with the stretched and penetrated body surface.

2. The method of claim 1, wherein the body surface comprises stratum corneum of skin.

3. The method of claim 1, comprising applying a tension of about 0.05 to about 2 M Pa to the penetrated body surface.

4. The method of claim 1, further including stretching the body surface during microprotrusion penetration.

5. The method of claim 1, wherein the the body surface is stretched in multiple directions.

6. The method of claim 1, wherein the agent is delivered over a period of at least one hour.

7. The method of claim 1, wherein the stretching is accomplished by adhering first and second portions of a stretching device to the body surface and moving the first and second portions apart.

8. The method of claim 7, wherein the first and second portions of the stretching device are moved apart by rotating a housing in a space between the first and second portions.

9 The method of claim 7, further comprising biasing the first and second portions apart.

10. The method of claim 9, further comprising removing a retainer which initially holds the first and second portions in a first position, and biasing the first portion away from the second portion when the retainer is removed.

11. The method of claim 1, wherein the stretching is accomplished by application of suction to the body surface.

12. The method of claim 1, wherein the micropathways are elongated microslits.

13. The method of claim 12, wherein the body surface is stretched in a direction transverse to the elongated microslits.

14. An apparatus for delivering an agent through a body surface having a plurality of micropathways therein, comprising:
a body surface stretching device having a body surface engaging first portion and a body surface engaging second portion;
means for moving the first portion away from the second portion, thereby applying a tension of about 0.01 to about 10 M Pa to the body surface to stretch the body surface; and
an agent containing reservoir positioned between the first portion and the second portion and in agent-transmitting relation with the stretched body surface and the micropathways.

15. The apparatus of claim 14, wherein the reservoir is in a wedge-shaped housing.

16. The apparatus of claim 15, wherein the wedge-shaped housing contacts an inclined surface on the first and/or the second portion to move the portions apart when the housing is inserted between the first and second portions.

17. The apparatus of claim 14, wherein the reservoir is in a rotatable housing which is rotated to move the portions apart.

18. The apparatus of claim 14, further comprising:
a retainer which initially holds the first portion and the second portion in a first position; and
a biasing member for urging the first portion away from the second portion when the retainer is removed.

19. The apparatus of claim 14, further comprising a holding member for holding the stretching device in an expanded state after the first and second portions have been moved apart.

20. The apparatus of claim 14, wherein the expandable device comprises:
a pair of elongated, spaced apart members connected by an expandable member;
an agent reservoir located between the elongated, spaced apart members; and
a spreader which is inserted between the elongated, spaced apart members.

21. The apparatus of claim 14, wherein the body surface engaging first portion and the body surface engaging second portion stretch the body surface in a direction transverse to a largest dimension of the pathways.

22. An apparatus for delivering an agent through a body surface, comprising:
a body surface stretching device having a body surface engaging first portion and a body surface engaging second portion;
means for moving the-first portion away from the second portion a predetermined distance to apply a tension of about 0.01 to about 10 M Pa to the body surface; and
an agent containing or collecting reservoir positioned between the first portion and the second portion and in agent-transmitting relation with the body surface, a body surface-contacting face of the reservoir having a plurality of microprotrusions.

23. The apparatus of claim 22, wherein the reservoir is located in a housing having a plurality of microprotrusions extending downward from a bottom of the housing.

24. The apparatus of claim 14, wherein the reservoir is in a wedge-shaped housing.

25. The apparatus of claim 15, wherein the wedge-shaped housing contacts an inclined surface on the first and/or second portion to move the portions apart when the housing is inserted between the first and second portions.

26. The apparatus of claim 14, wherein the reservoir is in a rotatable housing which is rotated to move the portions apart.

27. The apparatus of claim 14, further comprising:
a retainer which initially holds the first portion and the second portion in a first position; and
a biasing member for urging the first portion away from the second portion when the retainer is removed.

28. The apparatus of claim 14, further comprising a holding member for holding the stretching device in an expanded state after the first and second portions have been moved apart.

29. The apparatus of claim 14, wherein the stretching device comprises:
a pair of elongated, spaced apart members connected at their ends by an expandable member, the agent reservoir located between the elongated, spaced apart members; and
a spreader which is inserted between the elongated, spaced apart members.

30. The apparatus of claim 14, wherein the body surface engaging first portion and the body surface engaging second portion stretch the body surface in a direction transverse to a largest dimension of the pathways.

31. A stretching device for stretching a body surface having a plurality of micropathways formed therein, the stretching device comprising:
a member for contacting and at least partially surrounding a portion of the body surface in which the pathways are formed; and
means for applying a tension of about 0.01 to about 10 M Pa to the portion of the body surface.

32. The apparatus of claim 14 or 22, wherein the means for applying a tension includes a device for applying suction to the body surface.
